(19) United States
(12) Patent Application Publication
(10) Pub. No.: US 2021/0338195 A1
(43) Pub. Date: Nov. 4, 2021

(54) SMART HYDROGEL PILLAR AND FILM RESONATORS FOR BIOMEDICAL SENSING AND METHODS OF FABRICATION

(71) Applicant: UNIVERSITY OF UTAH RESEARCH FOUNDATION, Salt Lake City, UT (US)

(72) Inventors: Christopher F. Reiche, Salt Lake City, UT (US); Navid Farhoudi, Salt Lake City, UT (US); Florian Solzbacher, Salt Lake City, UT (US); Jules J. Magda, Salt Lake City, UT (US)

(21) Appl. No.: 17/366,743

(22) Filed: Jul. 2, 2021

Related U.S. Application Data
(63) Continuation-in-part of application No. 17/315,039, filed on May 7, 2021.
Application No. 17/315,039 is a continuation-in-part of application No. 16/330,048, filed on Mar. 1, 2019.
(60) Provisional application No. 63/047,884, filed on Jul. 2, 2020.
(60) Provisional application No. 63/022,098, filed on May 8, 2020.
(60) Provisional application No. 62/552,623, filed on Aug. 31, 2017.
(60) Provisional application No. 62/518,456, filed on Jun. 12, 2017.
(60) Provisional application No. 62/518,491, filed on Jun. 12, 2017.
(60) Provisional application No. 62/435,491, filed on Dec. 16, 2016.
(60) Provisional application No. 62/435,537, filed on Dec. 16, 2016.
(60) Provisional application No. 62/383,344, filed on Sep. 2, 2016.

(51) Int. Cl. A61B 8/08; A61B 90/00; A61K 49/22

(52) U.S. Cl. CPC A61B 8/0841; A61B 90/39; A61B 2090/3929; A61K 49/226; A61B 8/481

(57) ABSTRACT

Microresonator structures including a top polymer film layer, a bottom polymer film layer, and a smart hydrogel structure sandwiched between the polymer film layers. An ultrasound resonator cavity having a resonance frequency is defined between the top and bottom polymer layers, and the smart hydrogel structure is configured to provide a change in height to the ultrasound resonator cavity due to volumetric expansion or contraction of the smart hydrogel structure, in response to interaction of the smart hydrogel structure with one or more predefined analytes in an in vivo or other environment. Related methods of use for determining the presence or concentration of a given target analyte, as well as methods of fabricating such microresonator structures are also described.

### CROSS-REFERENCE TO RELATED APPLICATIONS

This application claims the benefit under 35 U.S.C. 119(e) of U.S. Provisional Patent Application Ser. No. 63/047,884, filed Jul. 2, 2020 and titled “FABRICATION PROCESS FOR FREE-STANDING SMART HYDROGEL PILLARS FOR SENSING APPLICATIONS”, which is herein incorporated by reference in its entirety.

This application is also a continuation-in-part under 35 U.S.C. 120 of U.S. patent application Ser. No. 17/315,039 filed May 7, 2021 and titled “IMPLANTABLE AND BIODEGRADABLE SMART HYDROGEL MICROMECHANICAL RESONATORS WITH ULTRASOUND READOUT FOR BIOMEDICAL SENSING”, which claims the benefit under 35 U.S.C. 119(e) of U.S. Provisional Patent Application Ser. No. 63/022,098, filed May 8, 2020 and titled “SMART HYDROGEL MICROMECHANICAL RESONATORS WITH ULTRASOUND READOUT FOR BIOMEDICAL SENSING”, each of which is herein incorporated by reference in its entirety. The present application is also a continuation-in-part of U.S. patent application Ser. No. 16/330,048, filed Mar. 1, 2019 and titled “ULTRASOUND IMAGING OF BIOMARKER SENSITIVE HYDROGELS, which is a U.S. National Stage Application under 35 U.S.C. 371 of PCT Application Serial No. PCT/US17/49944 filed Sep. 1, 2017, titled “ULTRASOUND IMAGING OF BIOMARKER SENSITIVE HYDROGELS”, which claims the benefit of (1) U.S. Provisional Patent Application No. 62/552,623, filed Aug. 31, 2017 and titled “HYDROGEL ULTRASOUND RESONATORS FOR BIOMARKER SENSING,” (2) U.S. Provisional Patent Application No. 62/518,456, filed Jun. 12, 2017 and titled “METHODS TO DETECT VOLUME CHANGES OF HYDROGELS USING ULTRASOUND,” (3) U.S. Provisional Patent Application No. 62/518,491, filed Jun. 12, 2017 and titled “METHODS TO DETECT VOLUME CHANGES OF HYDROGELS USING ULTRASOUND,” (4) United States Provisional Patent Application No. 62/435,491, filed Dec. 16, 2016 and titled “HYDROGEL ULTRASOUND RESONATORS FOR BIOMARKER SENSING,” (5) U.S. Provisional Patent Application No. 62/435,537, filed Dec. 16, 2016 and titled “NOVEL METHODS TO DETECT VOLUME CHANGES OF HYDROGELS USING ULTRASOUND,” and (6) U.S. Provisional Patent Application Ser. No. 62/383,344, filed Sep. 2, 2016 and titled “ULTRASOUND BASED TRANSDUCER MECHANISM FOR HYDROGEL SENSORS.” Each of the aforementioned is incorporated herein by reference in its entirety.

### STATEMENT REGARDING FEDERALLY SPONSORED RESEARCH OR DEVELOPMENT

This invention was made with government support under grant GM130241 awarded by the National Institutes of Health. The government has certain rights in this invention.

### BACKGROUND

### Technical Field

This disclosure generally relates to smart hydrogel structures for use in detecting a target analyte in a given environment, as well as methods for fabricating such structures.

### Related Technology

Advances in computing technology have resulted in a concomitant advance in medical device technologies, including within the field of diagnostic and interventional medicine. Particularly, the past century has demonstrated significant advances in medical imaging devices. Such advances have been hallmarked by the advent of radiologic devices such as computed tomography, magnetic resonance imaging, ultrasound, and other imaging devices that allow for the non-invasive viewing and exploration of internal structures of the body. These devices are often used with interventional radiology and minimally invasive surgeries as well, providing image guidance for any of a plethora of medical devices operated by a physician.

The non-invasive nature of medical imaging devices provide certain advantages, but they also have their limitations. Magnetic resonance imaging, for example, requires a patient to hold completely still in a confined area while the overly large, loud, and expensive imaging machine obtains image data. Other medical imaging devices, such as those used for medical ultrasound, are less expensive but often cannot provide high-resolution images of deep tissue sites.

Further, medical imaging devices are limited by the kind of information reported. Ultrasound, for example, generally relies on sonically reflective surfaces to produce an image and provides little information outside of the image data that can be derived from sonically reflective surfaces within the body. In some instances, ultrasound can be used to detect and monitor blood flow and heart rate, but ultrasound lacks the resolving power to identify the presence or absence—let alone the concentration—of biomarkers within the body. The other medical imaging techniques and devices available similarly lack the ability to identify biomarkers within the body, and while the medical imaging devices developed over the past century have allowed physicians and clinicians to better document, treat, and understand pathologies, they have their limits.

Accordingly, there are a number of disadvantages of existing systems and methods that can be addressed.

### BRIEF SUMMARY

Implementations of the present disclosure solve one or more of the foregoing or other problems in the art. One of the main challenges for implantable biomedical sensing schemes is obtaining a reliable and useful signal while at the same time maintaining biocompatibility. Applicant's U.S. application Ser. No. 17/315,039 filed May 7, 2021 and titled “IMPLANTABLE AND BIODEGRADABLE SMART HYDROGEL MICROMECHANICAL RESONATORS WITH ULTRASOUND READOUT FOR BIOMEDICAL SENSING”, herein incorporated by reference in its entirety, discloses use of a combination of medical ultrasound detection and smart hydrogel micromechanical resonators for continuous monitoring of biomarker or other analyte concentrations. The sensing principle is based on the shift of the mechanical resonance frequencies of smart hydrogel structures induced by their volume-phase transition in response to changing analyte levels. This shift is evident, and can be measured as a change in amplitude or intensity of an ultrasound query wave or pulse as induced by interaction of the one or more predefined analytes with the smart hydrogel microresonator structure in the environment being queried when the smart hydrogel biosensor is probed or queried by ultrasound at or near the resonance frequency. Similarly, such shift can be measured by tracking the frequency shift of the resonance peak (e.g., by changing the ultrasound query frequency to keep the phase of the signal constant). Such concepts eliminate the need for implanting complex electronics or employing transcutaneous connections for sensing biomedical analytes in vivo or in other environments. While such a change can be measured by observing change of contrast in an ultrasound image, such methods do not necessarily require generation of any ultrasound image, as all that is required is to track the change in ultrasound response (e.g., reflected or transmitted wave intensity or shift in resonance frequency), due to the change in resonance frequency of the smart hydrogel structure. It will be appreciated that in a perfect cavity the resonance frequency or frequencies would be integer multiples of half the wavelength, although of course no cavity is perfect.

Ultrasound as an imaging mechanism is limited in resolution, e.g., to about 0.5 mm. In addition, smart hydrogel structures have acoustic impedance characteristics that are very close to that of the surrounding environment, e.g., when used in vivo. Another difficulty is the relatively slow response time of such hydrogels, which response is diffusion dependent. Reducing the critical dimensions of the hydrogel structures can improve their response time. Such challenges can be addressed using ultrasound “readout” in combination with microresonator structures as disclosed herein, which exhibit geometries that can be easily manufactured, to include very small critical smart hydrogel structural dimensions, providing for reduced response time, without sacrificing sensitivity or mechanical stability.

Smart hydrogels are attractive for use as described herein due to their good biocompatibility and versatility. They can be tailored to selectively sense a variety of different analytes by employing different techniques such as molecular imprinting, incorporation of aptamers or inclusion of functional groups inside the polymer network that would be capable of reversible binding to a desired biomarker analyte. Such smart hydrogels may be formed from a hydrophilic network of polymers that experiences a change in volume and/or a change in other mechanical or other physical properties in response to specific stimuli, including contact with a desired analyte, such as glucose, another biomarker, or any other desired target analyte.

The present disclosure contemplates sensing of biomedical or other analytes based on resonance absorption of ultrasound in smart hydrogel microresonator structures that eliminates the need for contrast agents, and does not require any electrical or other signal connection exterior to the patient or other monitoring environment. The microresonator structure includes top and bottom polymer film layers, with a smart hydrogel sandwiched therebetween. A resonant cavity is defined between the polymer film layers, which cavity is dynamic, in that its volume and other dimensions (e.g., height) is determined by the swelling state of the smart hydrogel structure attached between the two polymer film layers. Any change in analyte concentration causes the smart hydrogel to swell or shrink, changing the resonance frequency of the resonant cavity defined between the polymer film layers. The approach as described herein remotely queries the microresonator structure using ultrasound waves, where the frequency of the ultrasound query is at or near a resonance frequency of the resonant cavity. While this can be used to detect analyte presence and/or concentration in the body as described herein, it can also be used in other environments, where detection of a target analyte is desired (e.g., in a petroleum or other product pipeline, or any other environment to be queried). The geometry of the smart hydrogel-based structure included in the microresonator structure may be any of a wide variety of geometries, including but not limited to pillars, walls, a continuous bulk hydrogel layer, or the like, where the smart hydrogel is sandwiched between the top and bottom polymer film layers.

The system can further include a computer system in electrical communication with the ultrasound transducer, the computer system having one or more processors, where the computer system is configured to receive ultrasound data (e.g., wave amplitude or intensity data) from the ultrasound transducer, such data being provided by query of the microresonator structure by the ultrasound transducer at or near the resonance frequency. The computer system is configured to determine (e.g., at the one or more processors) the change in amplitude or intensity of the ultrasound wave or pulse due to a change in the resonance frequency of the microresonator structure as induced by interaction of the one or more predefined analytes with the smart hydrogel structure of the microresonator structure. In addition or alternative to measuring change in wave or pulse amplitude or intensity, or shift in the resonance frequency itself, the system could be configured to measure a change in mean grayscale value (MGV) (e.g., in conjunction with B-mode ultrasound imaging), any of which parameters can be correlated to a concentration of the target analyte in the query environment.

In principle the whole measurement principle can be summarized as follows: 1) an ultrasound pulse of known frequency and amplitude is emitted by the transducer; 2) the pulse traverses the medium (e.g., body tissue, where losses occur (e.g., due to scattering, dampening, etc.), reducing the ultrasound wave amplitude; 3) the pulse reaches the microresonator structure, where the amplitude of the wave or pulse is changed (e.g., attenuated) according to the swelling state of the hydrogel structure in the resonator cavity (which attenuation correlates to the analyte concentration); and 4) after the pulse has traversed the microresonator structure and is reflected back or transmitted, the real-time status of the microresonator structure including its hydrogel structure is imprinted in this extra loss in amplitude. Once this pulse (in part or whole) reaches the detector (either in transmission or by reflection of the whole or part of the pulse) this information can be extracted by measuring the pulse amplitude or intensity with the timing of the pulse being used to determine if the pulse interacted with the microresonator structure or not. The reflected or transmitted wave amplitude after interaction with the microresonator structure is thus the key parameter that can be evaluated, either directly, or indirectly through another related parameter. Choice of query or probing frequency is important, and can strongly influence the resulting time-domain signal. Careful selection of query or probing frequency enhances the signal-to-noise ratio in the observed change in ultrasound wave amplitude or intensity, making selection of such query or probing frequency very important in an in vivo or similar complex, uncontrolled environment.

In an embodiment, the microresonator structure advantageously does not include any markers, contrast agents, or external connections, but is a simple sandwich structure as described herein. For example, the structure may consist or consist essentially of the hydrogel, and its associated top and bottom polymer film layers without any smart “chip” electrical components. Various structures that do not interfere with the biocompatibility and simplicity of the smart sensor may optionally be present.

In an embodiment, the smart hydrogel pillars, walls, or other structure sandwiched between the top and bottom polymer film layers can have a thickness from 50 μm to 1000 μm, from 100 μm to 1000 μm, or from 150 μm to 500 μm. The critical dimension (e.g., equal to the radius of a pillar, half the thickness of a wall, etc.) may be less than 500 μm, less than 300 μm, or less than 200 μm. The smaller the critical dimension, the faster the diffusion of the analyte into the smart hydrogel, and the faster the response time of the microresonator structure. The overall microresonator structure including the top and bottom polymer film layers with the smart hydrogel structure sandwiched therebetween may have a length and/or width from 0.1 mm to 20 mm, or 1 mm to 20 mm, or from 2 mm to 20 mm. In an embodiment, the polymer film layer can be intentionally perforated, thus creating a way to modify and tune its average effective acoustic impedance, e.g., where the holes are significantly smaller than the ultrasound wavelength, to improve the ultrasound signal. For example, holes formed through the thickness of the polymer film layer may serve to adjust ultrasound reflectance of the polymer film layer including such holes.

In an embodiment, any change in dimension or volume of the smart hydrogel structure or the microresonator structure as a whole as a result of interaction with the one or more predefined analytes in the in vivo or other environment may not necessarily be readily detectable in any ultrasound image itself, if any such image is even generated, as the scale of such change may be too small to be perceptible, given the limited resolution of ultrasound imaging. As described herein, generation of an ultrasound image is not actually required, as all that is needed is to measure the change in absorption of the ultrasound frequency used for the query, due to the analyte induced volumetric change in the smart hydrogel in the microresonator structure.

Associated methods of use are also disclosed, allowing a practitioner to monitor concentration of a given analyte, using the microresonator structures as described herein, implanted in the in vivo or other environment, as queried by the ultrasound transducer at or near the resonance frequency of the microresonator structure. Associated methods of manufacture are also described.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims, or may be learned by the practice of the disclosure as set forth hereinafter.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope.

In the drawings, multiple instances of an element may each include separate letters appended to the element number. For example, two instances of a particular element “100” may be labeled as “100a” and “100b.” In that case, the element label may be used without an appended letter (e.g., “100”) to generally refer to every instance of the element, while the element label will include an appended letter (e.g., “100a”) to refer to a specific instance of the element. Similarly, a drawing number may include separate letters appended thereto. For example, FIG. 1 may include FIG. 1A and FIG. 1B. In that case, the drawing number may be used without the appended letter (e.g., FIG. 1) to generally refer to every instance of the drawing, while the drawing label will include an appended letter (e.g., FIG. 1A) to refer to a specific instance of the drawing. The disclosure will be described and explained with additional specificity and detail through the use of the accompanying drawings in which:

FIGS. 1A-1B illustrate a plan view and an elevation view of pillar hydrogel structures, including exemplary dimensions according to one example.

FIG. 2A is a schematic view of a hydrogel lithography assembly process that can be used to fabricate hydrogel pillars or other structures.

FIG. 2B is an optical image of a fabricated smart hydrogel pillar after peeling off the polyimide film from the shadow mask. Such hydrogel structures can be kept hydrated to avoid damage.

FIG. 3 charts change in MGV of exemplary smart hydrogel pillar structures on a 25 μm thick polyimide film as compared to the same 25 μm thick polyimide film without any hydrogel structure thereon, as compared to a 279 μm thick continuous hydrogel bulk sheet on a 25 μm thick polyimide film.

FIGS. 4A-4G schematically and progressively illustrate an exemplary method for fabricating a microresonator structure as described herein, including top and bottom polymer film layers, with a smart hydrogel structure sandwiched therebetween.

FIG. 5A schematically illustrates how the polymer film layer(s) can optionally be surface treated to better adhere to the smart hydrogel, e.g., between the steps shown in FIGS. 4B and 4C.

FIG. 5B schematically illustrates where if perforation of the top polymer layer is desired, such can be accomplished between the steps shown in FIGS. 4B and 4C.

FIG. 5C schematically illustrates how the desired smart hydrogel structures can be formed via two-photon 3D lithography, rather than a photomask-based approach.

FIG. 5D schematically illustrates the optional addition of metal reflective layer(s) over the polymer layers after the step shown in FIG. 4B.

FIG. 5E schematically illustrates optional structuring of the applied metal reflective layer from FIG. 5D.

FIG. 5F schematically illustrates addition of an additional polymer film layer.

FIG. 5G schematically illustrates how photolithography may be performed through a transparent bottom substrate to transmit light for hydrogel polymerization, rather than through the top layer.

FIG. 5H schematically illustrates how a sacrificial or adhesion reducing layer can be provided, e.g., prior to the step shown in FIG. 4B, to aid in delamination of the substrate(s). When a sacrificial or anti-adhesive layer is used, the fabrication process can be used to create free standing pillars on a thin polymer substrate film layer, such as shown in FIG. 1B. Such structures can be used as resonator structures themselves, e.g., as described in parent patent application Ser. No. 17/315,039, incorporated by reference in its entirety herein. The sandwich structures described herein are an additional application of this manufacturing technique, providing a potentially further improved and more versatile resonator structure.

FIG. 6 schematically illustrates the present ultrasound read-out mechanism for using the present microresonator structures, where ultrasound waves are generated by the probe, and the reflections (or transmitted waves) are recorded by the receiver element(s). The intensity and timing of the returning waves in each spatial location are used to determine wave attenuation, which can be correlated to the concentration of the target analyte in the environment where the microresonator structure is placed.

FIG. 7A illustrates an example of the present microresonator structure including a perforated top polymer film layer for increased ultrasound transparency.

FIG. 7B illustrates another example of the present microresonator structure including a combination of smart hydrogel pillars and walls between the top and bottom polymer layers, where the corner walls provide for increased stability.

FIGS. 8A-8F illustrate additional examples of possible microresonator structures, shown without the top polymer film layer in place, to better illustrate the interior smart hydrogel structures.

FIG. 8A illustrates a microresonator structure including an array of pillars sandwiched between the top and bottom polymer film layers.

FIG. 8B illustrates a microresonator structure including a series of walls sandwiched between the top and bottom polymer film layers.

FIG. 8C illustrates a microresonator structure including an array of geometric crosses sandwiched between the top and bottom polymer film layers.

FIG. 8D illustrates a microresonator structure including a combination of pillars and walls sandwiched between the top and bottom polymer film layers.

FIG. 8E illustrates a microresonator structure including a porous smart hydrogel bulk layer sandwiched between the top and bottom polymer film layers.

FIG. 8F illustrates a microresonator structure including a smart hydrogel peripheral wall defining an interior cavity, sandwiched between the top and bottom polymer film layers.

### DETAILED DESCRIPTION

Before describing various embodiments of the present disclosure in detail, it is to be understood that this disclosure is not limited to the parameters of the particularly exemplified systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments, and is not necessarily intended to limit the scope of the claimed invention.

### Ultrasound and Hydrogel Sensing

As discussed above, medical imaging devices are generally limited in the kind information that can be reported. Ultrasound, for example, generally relies on sonically reflective surfaces to produce an image and provides little information outside of image data. Normally, ultrasound lacks the resolving power to identify the presence or absence—let alone the concentration—of a biomarker or other analyte within the body or in any other environment. The combined use of ultrasound and microresonator structures that incorporate smart hydrogels as described herein, however, can be adapted for this purpose. As described herein the presently contemplated solutions can be a standalone sensing solution with a corresponding ultrasound transducer that does not require the generation of an image or even calculations associated with image generation. Rather, as described herein, the present methods can be achieved more directly, based on ultrasound pulse/wave detection (particularly changes in intensity or amplitude, or tracking of the shift in resonance frequency).

Hydrogels are structures that include hydrophilic cross-linked networks of polymer that have both liquid-like and solid-like properties. Smart hydrogels characteristically experience a change in their volume and/or mechanical properties in response to the presence of a specific stimulus or analyte, particularly where the hydrogel incorporates functional groups that can reversibly bind to the target analyte. For example, aptamers (e.g., short single strands of nucleic acids such as DNA or RNA) can be incorporated into the hydrogel, allowing it to selectively bind to target biomarkers (e.g., glucose, proteins, other peptides, opioids, other drugs or drug metabolites to be detected, etc.) or other target analytes to allow the smart hydrogel to serve as a identifier of whether and how much of the target analyte is present.

As used herein, the term “analyte” is to be construed broadly, and includes any substance that can itself be identified or measured or of which a chemical or physical property thereof can be identified or measured. Analytes include, for example, nucleic acids, proteins, other peptides, or other compounds. Glucose is a specific example of an analyte. In some instances, analytes serve as a physiologic, pathologic, or environmental markers of a known or unknown phenomenon (e.g., glucose or insulin levels can serve as a biomarker for diabetes). It should be appreciated that the disclosed embodiments apply generally to smart hydrogels that are responsive to any desired target analyte, whether in the context of treatment of a human or other patient, or in other contexts where analyte detection and concentration measurement would be useful (e.g., pipeline or other environment monitoring or the like). A key advantage of such embodiments is that remote measurement without any cables or other connections is possible, so that a pipeline or other barrier does not need to be breached, which introduces a weak point in such a system.

Hydrogels can also respond to the presence of an environmental stimulus (e.g., temperature, pH, gas, osmolarity, humidity, etc.) and can additionally serve to indicate particular state data of an aqueous solution, such as pH. That is, hydrogels can change their volume and/or mechanical properties in response to the level of salinity or acidity in an aqueous solution. The present systems and methods can be used to detect and measure such.

A hydrogel can transition from a collapsed or shrunken state to a swollen state in response to the presence (or absence) of a specific analyte. Such a change is typically not binary, but the degree of change is gradual, depending on the concentration of analyte present in the environment of interest. Of course, the concentration of the analyte can span any particular concentration along a spectrum of concentration values, from relatively low, to relatively high. Thus, in other words, the change in volume of the hydrogel due to the presence of the target analyte can correlate to the concentration of the analyte, and the system can be calibrated to provide such analyte concentration data to a user of the system, based on the changes to the microresonator structure including the smart hydrogel.

The hydrogel is configured to swell or otherwise change volume (e.g., shrink) in response to interaction with the target analyte in response to the concentration of analyte present. The biomarker sensitive hydrogel can be configured to reach an equilibrium within a given time period based on the concentration of analyte available, and the critical dimensions of the given smart hydrogel structure.

To now, the ability to obtain a real-time, visual readout of hydrogel responses to analytes or other stimuli has proven problematic, particularly when the hydrogel is implanted in vivo or in a similarly demanding environment. Noninvasive medical imaging techniques would be an ideal method to obtain a real-time, visual readout of hydrogel responses in vivo, but hydrogels are normally nearly invisible to most medical imaging devices—including ultrasound—making it difficult to determine any response of the hydrogel to surrounding analytes and/or stimuli. Thus, even though hydrogels represent a promising material for biomedical and biotechnological applications, their lack of visibility and concomitant lack of ability to be tracked in real time using current imaging devices and techniques has made their potential unrealized.

The present disclosure provides a novel approach for sensing of biomedical analytes based on resonance absorption of ultrasound in microresonator structures that incorporate a smart hydrogel structure, where changes in the smart hydrogel structure cause a change in an ultrasound resonator cavity defined by the microresonator structure, where the change in height or other dimension of the resonator cavity results in a change in resonance frequency of such cavity. Such a change can be detected without use of any contrast agents. This approach uses a mechanical microresonator structure that may include top and bottom polymer film layers, with a smart hydrogel structure sandwiched between such layers, so that a resonator cavity is defined between such polymer film layers. Because the smart hydrogel can expand or contract upon interaction with the target analyte, any such change will alter the height or other dimensions of the resonator cavity, and hence the cavity's resonance frequency. A change in the resonance frequency of the cavity affects the degree to which an ultrasound wave or pulse is attenuated upon interaction with such cavity, where such ultrasound wave or pulse used to make the query is at or near the resonance frequency of the cavity. This degree of attenuation can be detected, and can be correlated to the concentration of the target analyte in the environment in which the microresonator structure is placed.

### Sensor Concept

In B-mode (also known as 2D mode) ultrasound imaging, a linear array of transducers sends and receives ultrasound waves to and from a medium to create a 2D image based upon the timing and intensity of the incident and reflected waves. While such 2D mode ultrasound imaging can be used as described herein to make the query, it will be appreciated that other modes (e.g., pulse echo mode or others) of ultrasound devices may also be suitable for use. In 2D mode imaging, the reconstructed 2D image represents a 2-dimensional cross-section of the medium, and the intensity of each pixel in the image is the logarithmic ratio between the intensities of the incident and reflected waves from the corresponding spatial point. The change of acoustic impedance when transmitted between two media types determines the amount of the ultrasound signal that is reflected from the boundary. Boundaries with closely matched acoustic impedances do not exhibit considerable contrast in the ultrasound image. As the acoustic impedances of hydrogel and the surrounding aqueous medium are very similar due to the high water content of the hydrogel, the intensity of the reflections from the hydrogel/solution boundary is very small. Therefore, using ultrasound imaging directly to assess the swelling state of hydrogel provides only limited information.

To solve this challenge, the present disclosure uses microresonator structure geometries that are specifically patterned to define an ultrasound resonator cavity, where a smart hydrogel structure spans the height of such cavity, and a geometric change in the smart hydrogel structure causes a change in the ultrasound resonator cavity, which causes a change in the cavity's resonance frequency. Such a microresonator structure can include top and bottom polymer film layers, with a smart hydrogel structure positioned between the polymer layers, so as to define a resonator cavity between such polymer layers. The distance between such polymer layers defines the resonator cavity, and any swelling or shrinking of the smart hydrogel within the cavity results in an expansion or shrinking of such cavity, affecting the resonance frequency associated with the cavity. The smart hydrogel between the polymer film layers may have any of various geometries, e.g., an array of pillars, walls, a continuous bulk layer that is substantially transparent to the ultrasound query frequency (so that a resonator cavity is defined within such space), or the like. Such microresonator structures may enhance the response time needed to detect a change in analyte concentration, as response time depends on the equilibrium saturation of the smart hydrogel with the analyte, and reducing the critical dimensions of the smart hydrogel allows response time to be accelerated. The presence of the top and bottom polymer film layers provide the overall structure with strength and durability, even though the smart hydrogel portion of the microresonator structure itself may be a relatively thin elongate structure, if separated from the polymer film layers to which the smart hydrogel structure is attached.

Ultrasound waves are mechanical compression waves, and as such can excite mechanical vibrations in structures they pass through. When the microresonator structures described herein are probed by ultrasound waves having a frequency close to the cavity's resonance frequency, a high fraction of the mechanical energy from the ultrasound wave or pulse is absorbed within the cavity. This lowers the reflected or transmitted ultrasound wave or pulse intensity and thus creates additional contrast in any generated ultrasound image, even where there may be a close acoustic impedance match with the surrounding environment. Of course as noted herein, the present methods and systems do not actually require generation of an image, but merely the measurement of the change in ultrasound wave intensity.

Any change to the volume or other geometric dimensions of the smart hydrogel structures alters the resonance frequency of the cavity defined by the microresonator structure and, therefore, the amount of energy from the ultrasound waves that is absorbed. The amount of absorption depends on the frequency separation between the resonance frequency and the excitation frequency (i.e., the ultrasound waves used for making the query), such that the closer the query frequency is to the peak resonance frequency, the more pronounced the effect will be. This resonance induced absorption changes the ultrasound wave intensity being transmitted or reflected back to the ultrasound transducer. This concept of using resonance frequency of a resonator cavity of the microresonator structure for the query enables the measurement of small changes in the microresonator structure induced by changing analyte concentrations, even where the microresonator structure or the smart hydrogel components thereof are so small that the change due to swelling or shrinking may not be ascertainable in any ultrasound image that may be produced. Indeed, as described herein, no image at all even need be generated or displayed for the present methods and systems to work.

In general, any mechanical resonance mode of the cavity that can be excited by ultrasound can be employed for the present methods. Possible mechanical resonance modes of a given microresonator structure depend on the geometry of such structure. If the frequency of the ultrasound waves is close to (e.g., within 30%, within 25%, within 20%, within 10%, or within 5% of) a resonance frequency of the cavity, the corresponding resonance mode can be excited. Of course, such may depend on the resonator quality factor.

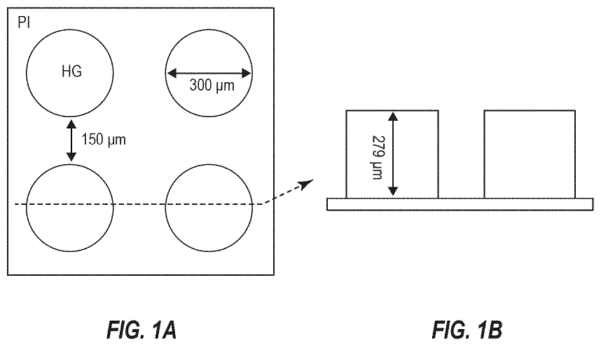

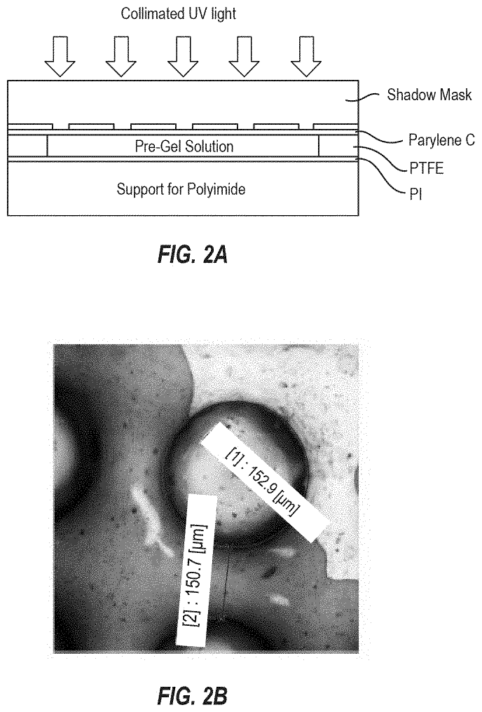

In some embodiments, the microresonator structure can be relatively small, e.g., less than 1 mm, or even less than 0.5 mm thick so as to be capable of injection into a desired location through a narrow gauge needle, or similar implantation technique. In an embodiment, the microresonator structure may be elongate in shape, such that the width or diameter of such is disproportionate to its length, although a wide variety of shapes and geometries are possible, any of which may be well suited for providing a resonant cavity with a resonance frequency within the desired range. For example, the cavity is really just the top and bottom surfaces of the polymer film, that are ideally parallel to each other, and although the shape of the cavity is illustrated in the Figures as rectangular, it could be a circle, square or irregularly shaped. The smart hydrogel structure within the microresonator structure may also have relatively small dimensions, e.g., to ensure a fast response time, for the smart hydrogel structure to substantially reach an equilibrium state when exposed to an environment including the target analyte. In an embodiment, a given portion of the hydrogel structure (e.g., diameter, width, or height of a pillar, wall, or the like) can have a thickness greater than 5 μm, greater than 10 μm, greater than 20 μm, greater than 30 μm, greater than 40 μm, greater than 50 μm, greater than 70 μm, greater than 80 μm, greater than 90 μm, greater than 100 μm, greater than 150 μm, greater than 200 μm, less than 1000 μm, less than 500 μm, less than 400 μm, less than 300 μm or less than 200 μm. For pillars or walls, spacing between adjacent structure may be in a similar range. By way of example, exemplary pillars, walls or the like (e.g., positioned between top and bottom polymer film layers, defining a resonator cavity between the polymer film layers) may have a height of from 10 μm to 1000 μm or from 50 μm to 500 μm, may have a diameter or thickness of from 30 μm to 500 μm, or from 50 μm to 300 μm, and a spacing between pillars or walls of 1 μm to 500 μm, or from 5 μm to 300 μm. Critical dimensions of such smart hydrogel structures may generally be half of the above values, e.g., to correspond to the radius of a pillar or half the thickness of a wall, which is the distance that an analyte must diffuse through, for the smart hydrogel to reach an equilibrium state. Such values are of course merely provided as examples. FIGS. 1A-1B illustrate exemplary hydrogel pillars. Such pillars could be positioned between polymer film layers (e.g., polyimide or any other biocompatible material with high enough acoustic impedance) as described herein. The illustrated configuration includes pillars having a diameter of 300 μm, a height of 279 μm, and spacing between adjacent pillars of 150 μm. The illustrated bottom polyimide film layer has a thickness of 25 μm. A top polyimide film layer could similarly have a thickness of 25 μm. Another example is illustrated in FIG. 2B, where the pillar has a radius of 152.9 μm and spacing between adjacent pillars of 150.7 μm. In general the thickness of the microresonator structure, particularly the resonator cavity thereof, may be adapted to the ultrasound frequency used for query and may typically be from about 25% to about 100% of the ultrasound wavelength in the hydrogel or other applicable medium, where it is intended to excite the longitudinal or other resonance associated with such cavity.

### Hydrogel Sensor Fabrication

Early smart hydrogel structures prepared by Applicant included hydrogel micropillars and hydrogel sheets. In some cases, there was no significant improved response time of the hydrogel micropillars as compared to a hydrogel sheet, as the early employed mold-based microfabrication process only allowed for a small pillar height and required the pillars to be attached to a comparatively thick hydrogel backplane. Therefore, the response of this hydrogel structure was dominated by the relatively hydrogel backplane as it also needed to reach equilibrium.

Consequently, a fabrication technique was developed that provides cost-efficient and reliable fabrication of microscale free-standing smart hydrogel microresonator pillars. In addition to this, experiments have been conducted showing the ability of such pillars to sense changes in ionic concentration. These preliminary results demonstrate an improved response time when compared to a simple hydrogel sheet, due to replacement of the previous hydrogel backplane structure (which slowed response time) with a thin polymer film layer that is not sensitive to the analyte.

A sheet of hydrogel with a thickness of 279 μm shows a good ultrasound response at 4 MHz and 9 MHz. This response appears to be the result of longitudinal standing waves inside the structures. For the sake of comparability, the height of the pillars in this study was also chosen to be 279 μm, although other heights could be selected. The geometry of the pillars used in one example is shown in FIGS. 1A-1B. The hydrogel pillars had a diameter of 300 μm, with spacing between adjacent pillars of 150 μm, as shown. The critical diffusion dimension for such a geometry is also 150 μm, equal to the radius of the pillars. The pillars were fixed on a 25 μm thick polyimide (PI) film.

For the fabrication of the hydrogel pillars, the PI films were functionalized to promote adhesion of the hydrogel to the film. A pre-gel solution was prepared as described in the inventors' publications N. Farhoudi, H.-Y. Leu, L. B. Laurentius, J. J. Magda, F. Solzbacher, and C. F. Reiche, “Smart Hydrogel Micromechanical Resonators with Ultrasound Readout for Biomedical Sensing,” ACS Sens., June 2020, doi: 10.1021/acssensors.9b02180 and N. Farhoudi, L. B. Laurentius, C. F. Reiche, J. Magda, and F. Solzbacher, “Micromechanical Resonators for Ultrasound-Based Sensors,” Meet. Abstr., vol. MA2020-01, no. 31, p. 2328, May 2020, doi: 10.1021/acssensors.9b02180, and N. Farhoudi, H.-Y. Leu, J. Magda, F. Solzbacher, and C. F. Reiche, “A Biomedical Sensor Based on Resonant Absorption of Ultrasound Waves in Hydrogel-based Resonators,” TechConnect Briefs, vol. TechConnect Briefs 2019, and had the same vacuum treatment for the sake of comparability. The prepared pre-gel solution contained acrylamide and bis-acrylamide monomers and a boronic acid-containing functional group that can be cross-linked by free radical copolymerization using a UV initiator. The resulting smart hydrogel after being exposed to UV light is responsive to changes in glucose concentration, pH, and ionic strength. The pre-gel solution was poured over the PI film, and then a 279 μm thick polytetrafluoroethylene (PTFE) spacer was placed over the film surrounding the solution. Next, a polyester dark field shadow mask, coated with a thin layer of Parylene C to avoid smart hydrogel adhesion, was placed over the solution and pressed on to seal the solution between the mask and the PI film. Subsequently, the sandwich was exposed to a collimated UV light source with a wavelength of 365 nm. The PI film was then gently removed from the mask and stored in phosphate-buffered saline (PBS). FIG. 2A schematically illustrates the assembly used to expose the pre-gel solution over the PI film. FIG. 2B shows an optical microscopy image of a resulting hydrogel pillar after peeling off the PI film from the mask. The pillar of FIG. 2B has a radius and spacing between adjacent pillars of about 150 μm.

The fabricated pillars went through a series of conditioning steps to remove the unpolymerized chemicals that are physically trapped inside the hydrogel network during polymerization to ensure a repeatable response from the hydrogels.

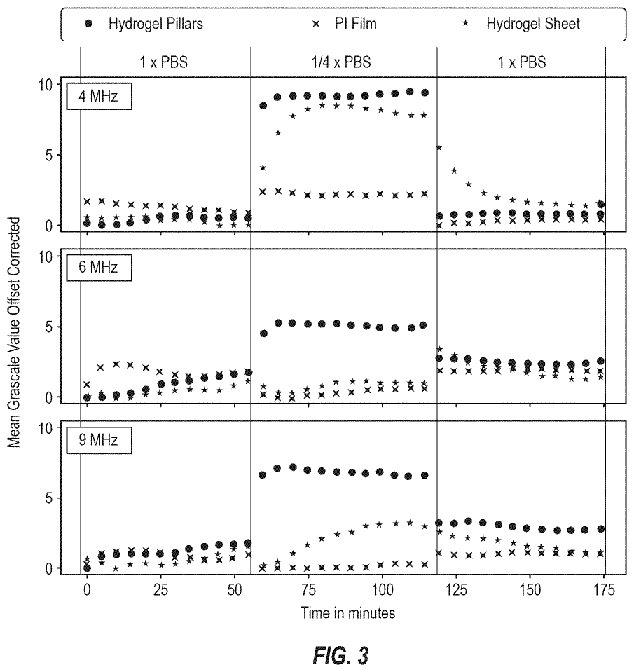

Three test samples were prepared. The first test sample was the fabricated hydrogel pillars with the geometry as described above. The second test sample was a control PI film with a thickness of 25 μm. The third test sample was a 279 μm thick hydrogel sheet attached to a 25 μm PI film. All the samples were cut into 8 mm diameter circular discs and mounted on a 3D printed polylactic acid (PLA) sample holder. These were then immersed into the testing solutions and imaged every 5 minutes using a medical ultrasound imaging system (ACUSON S2000, Siemens Medical Solutions USA, Inc.) and an array probe (9L4, Siemens Medical Solutions USA, Inc.). The obtained images were analyzed. The acquired grayscale images were stabilized using software to compensate for any rotation and translation of the ultrasound probe relative to the samples. Then a box of 20×100 pixels was selected on the boundary of the PLA and the surrounding solution, which contains the hydrogel and PI samples. The 8 bit (0 to 255) mean grayscale value (MGV) of the selected box was recorded over time, and an offset equal to the minimum value of the whole dataset was subtracted. FIG. 3 shows the measurement results for the three test samples at 4, 6, and 9 MHz imaging frequencies. Exemplary ultrasound query frequencies used in the present methods may be any desired value, such as from 2 to 20 MHz, from 4 to 10 MHz or from 4 to 6 MHz.

The fabrication technique as described enables the fabrication of a large number of hydrogel pillars in a cost-effective and fast manner. In addition, this technique does not rely on a microfabrication facility and can be done in a simple laboratory setup. The sizing of the pillars was verified using optical microscopy, which showed a high degree of uniformity across the fabrication area. Note that the fabrication area could be easily extended to produce larger arrays by increasing the area of collimated UV light and the size of the mask.

For the case of the PI film control sample shown in FIG. 3, the MGV is comparatively stable throughout the experiment, although it experiences an overall step when the solution is exchanged to a different level of ionic concentration (e.g., 1×PBS vs. ¼×PBS). This could be attributed to the change in the acoustic impedance of the surrounding test solution for different ionic strengths. The signal magnitudes of both the pillars and the sheet are substantially higher and show a time-dependent behavior that is characteristic for smart hydrogel-based sensing. For the case of the 279 μm hydrogel sheet, the MGV increases as the ionic concentration decreases, which is the result of swelling in the smart hydrogel. The response reaches an equilibrium in less than an hour. For the case of the hydrogel pillars, while showing the same signal trend, the response speed is significantly faster than for the hydrogel sheet. The faster response of the hydrogel pillars is due to the increased surface contact area and reduced critical dimension of the hydrogel pillars (˜150 μm radius vs. 279 μm thickness for the sheet), which increased the ionic exchange and shortens the diffusion time of the ions into the structure. In FIG. 3, one observes almost a step response in the signal for pillars as they shrink when placed in 1×PBS. The response time for swelling is slower than for shrinking and the shrinking response may have been simply too fast for the limited time-resolution of the experiment.

Finally, the signal amplitude is the highest at 4 MHz and is the lowest at 6 MHz. The response amplitude at 9 MHz, especially for the sheet, may have been affected by the presence of air bubbles around the sample.

Overall, these limited preliminary experiments, especially the results at 4 MHz, indicate that the above-described fabrication process can be used to create free-standing smart hydrogel pillars that can be used as sensing elements in an ultrasound readout scheme. Furthermore, these results also suggest an improved response time due to the reduced critical dimension of these pillars.

The described fabrication process can be used to fabricate free-standing hydrogel pillar-like structures. Furthermore, results indicating that smart hydrogel pillar microresonator structures fabricated according to such process can be used as a sensing element with an ultrasound-based readout scheme. These preliminary results also demonstrate an improvement of the response time of the hydrogel structures compared to larger flat hydrogel sheets due to the reduced critical dimension for diffusion.

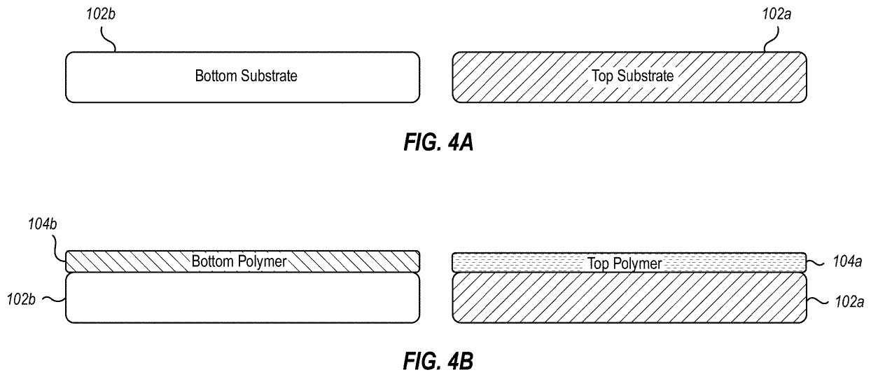

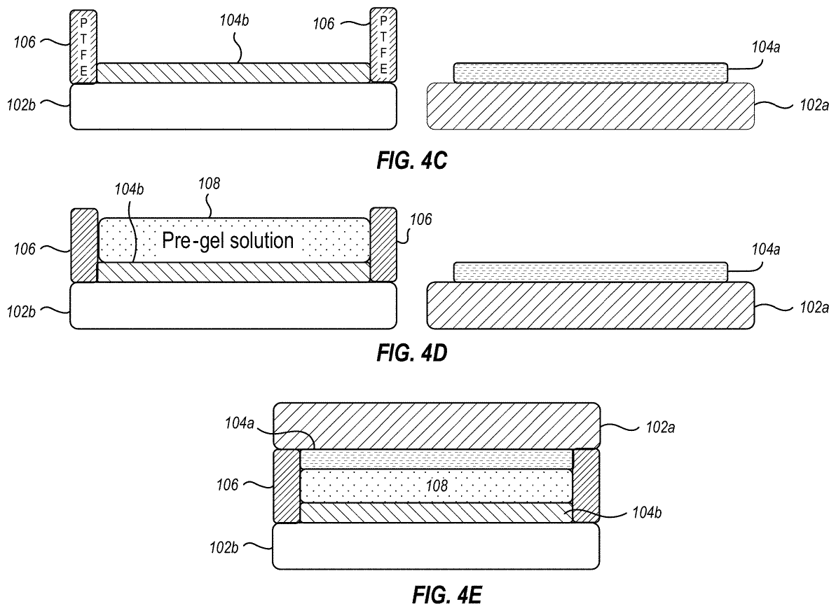

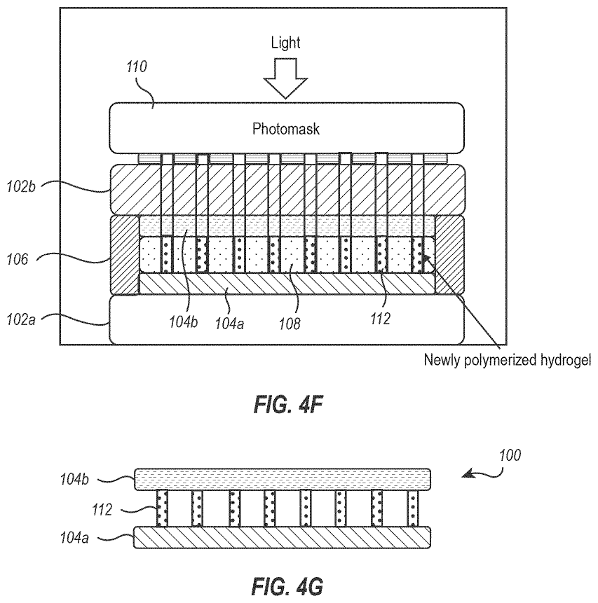

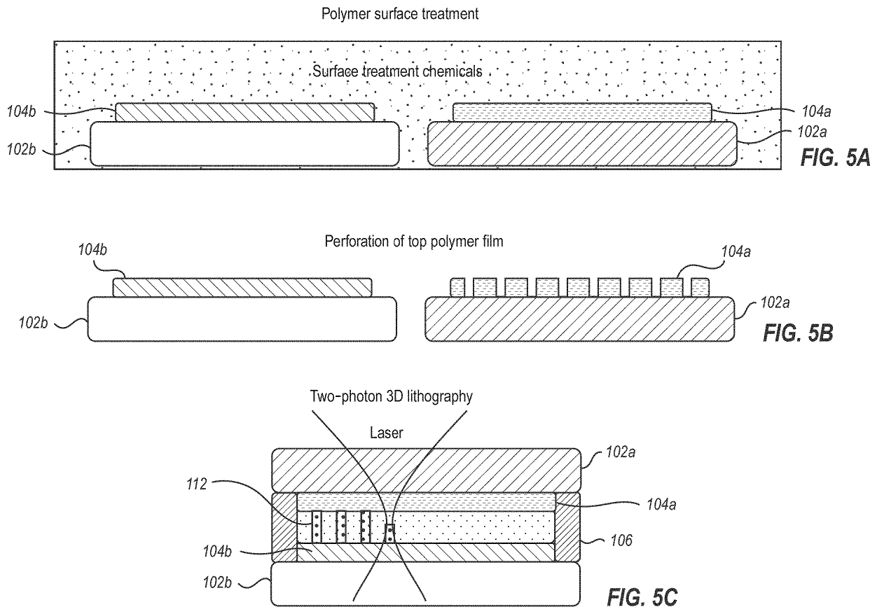

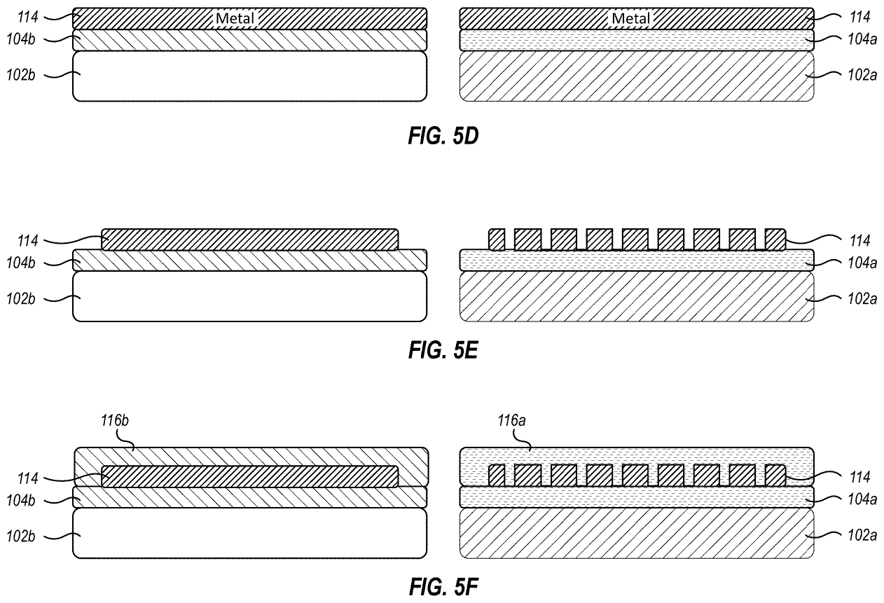

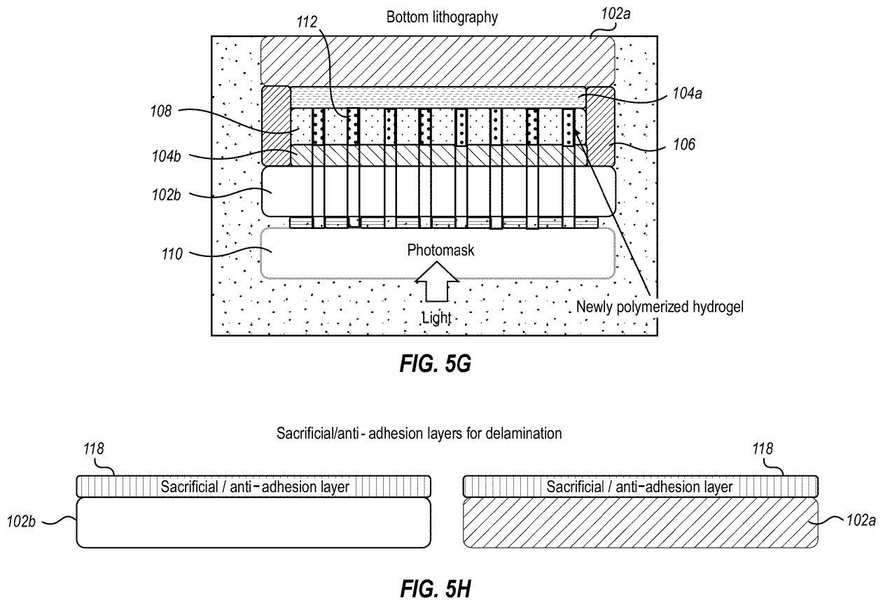

FIGS. 4-5 illustrate additional fabrication methods that may be used to form not just free standing hydrogel pillars on a thin polymer film layer, but sandwich structures including both top and bottom polymer film layers (e.g., polyimide or other polymer), with smart hydrogel structure(s) sandwiched therebetween, to create a resonant cavity therein. Such structures may provide the benefits noted above relative to faster response times, while also maintaining good mechanical stability for the resonator structure as a whole.

As shown in FIG. 4A, such a process may begin with two substrates (e.g., top and bottom substrates 102a, 102b). One substrate may be silicon, glass or any other microfabrication substrate material. The other substrate is specifically selected to be UV transparent, to allow UV curing of the hydrogel structure therethrough. In an embodiment, both such substrates 102a and 102b may be UV transparent.

As shown in FIG. 4B, both substrates 102 are coated with a polymer film 104 (e.g., polymerization from a precursor solution, lamination, or the like). The illustrated top and bottom polymer film layers 104a, 104b may be the same, or different polymer materials. After such deposition of the polymer film layer, the polymer films can be structured to provide any desired sensing device outline (e.g., rectangular, circular, oval, or any other desired shape. Such can be achieved with photolithography and either wet or dry etching methods. By way of example, a perimeter edge portion of the polymer film layer may be removed, in order to make way for a PTFE spacer element.

As shown in FIG. 4C, the spacer element 106 (e.g., PTFE or similar material) can be positioned around one of the polymer film layers 104a, 104b (FIG. 4C shows the spacer element positioned around the perimeter of the bottom film layer 104b). Such spacer element 106 may have a height similar to the thickness of the final microresonator structure 100. The spacer element 106 effectively forms a reservoir into which the pre-gel solution 108 can be filled, as shown in FIG. 4D. As shown in FIG. 4E, the top polymer layer 104a may then be positioned and aligned over the spacer element 106 and pre-gel solution 108.

As shown in FIG. 4F, a photomask 110 may be added and aligned over the assembly of FIG. 4E, and the pre-gel solution 108 may be exposed to collimated curing light wavelengths (e.g., UV), polymerizing the pre-gel 108 so as to form the smart hydrogel 112. The finished microresonator sandwich structure 100 as shown in FIG. 4G may be released by carefully peeling or otherwise delaminating the adjacent structures by removing the top and bottom substrates 102a, 102b, leaving behind the top and bottom polymer film layers 104a, 104b, with the smart hydrogel structure 112 positioned therebetween. Any unreacted pre-gel 108 may be removed through rinsing of the structure 100, followed by conditioning. The microresonator structure 100 may be maintained in a hydrated conditioned state until ready for use.

FIGS. 5A-5H illustrate various optional steps or features that may optionally be included in the fabrication method described in conjunction with FIGS. 4A-4G. For example, as shown in FIG. 5A, the polymer film 104 may be surface treated to adhere to the smart hydrogel 112. Such a step may be performed between the steps shown in FIGS. 4B and 4C. As shown in FIG. 5B, if perforation of the top polymer film layer 104a (or bottom polymer film layer 104b) is desired, such can be performed in the structuring step described as following the configuration seen in FIG. 4B, or can be performed using an appropriate photolithography mask, or may be a separate step performed prior to the state seen in FIG. 4C.

As shown in FIG. 5C, instead of using a photomask approach as shown in FIG. 4F, the desired smart hydrogel structures 112 may be formed via two-photon 3D lithography.

As shown in FIG. 5D, metal or other ultrasound reflective coating or layer(s) 114 may be added to the polymer films 104a, 104b, e.g., simply as a metal deposition step after the step shown in FIG. 4B. Such addition of reflective coatings or layers may be used to freely adjust the acoustic impedance of the polymer film layer, e.g., if the polymer alone is not acoustically different enough from the surrounding environment. FIG. 5E shows that to allow subsequent structuring of the polymer film, either or both of the metal or other reflective coatings or layers 114 can be structured as well. Such can be achieved through photolithography in combination with wet or dry etching techniques. Any desired holes for photo-exposure in the top film 104a can be added during such step as well, if desired.

FIG. 5F shows optional addition of a second polymer layer 116a, 116b over the metal or other reflective coating or layer 114. After such optional addition of a second polymer layer 116a and/or 116b (e.g., for protection of the reflective coating or layer 114), the fabrication process may continue as shown in FIG. 4C.

FIG. 5G shows how curing of the hydrogel structures may be achieved through a transparent bottom substrate, e.g., if for some reason the top layer is not or cannot be used to transmit light for hydrogel polymerization. Finally, FIG. 5H shows how if delamination of the structure in FIG. 4G proves to be difficult, a sacrificial or adhesion reducing layer 118 (e.g., Parylene C) could be added prior to the step associated with FIG. 4B, to help with any such issues.

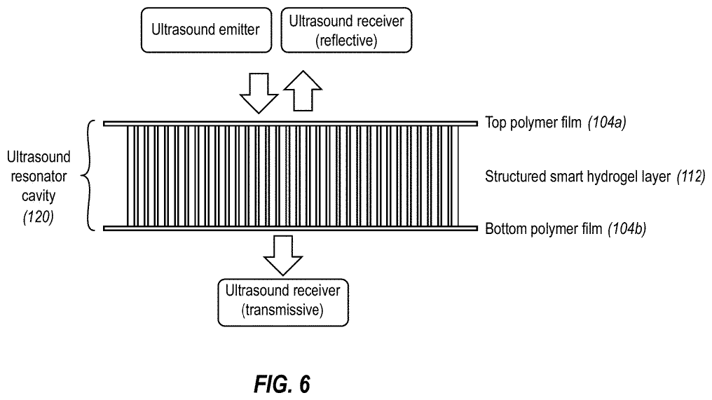

FIG. 6 schematically illustrates the present ultrasound read-out mechanism for using the present microresonator structures 100, where ultrasound waves are generated by the probe, and the reflections (or transmitted waves) are recorded by the receiver element(s). The intensity and timing of the returning waves in each spatial location are used to determine wave attenuation, which can be correlated to the concentration of the target analyte in the environment where the microresonator structure 100 is placed. As shown, the waves received by the receiver element(s) can either be reflected and/or transmitted, e.g., depending on the properties of the microresonator structure that the ultrasound waves or pulses interact with. In an embodiment, ideally, pulses, rather than waves may be used for the ultrasound query, as pulses give the cavity time to dissipate collected wave energy (by emission or liquid damping) in between measurements. For example, a fully excited resonator will no longer accept energy and will thus create only a small signal. Therefore, although a continuous wave may be possible, the use of discontinuous waves, or pulses may be more beneficial.

### Microresonator Sandwich Structures

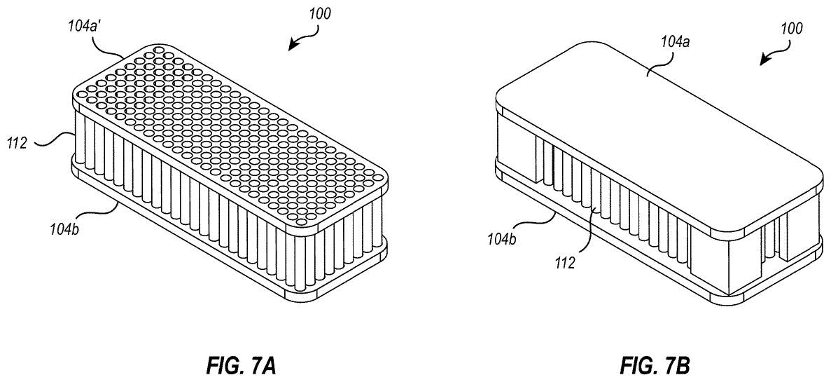

The mechanical microresonator structure 100 as shown in FIG. 4G includes a structured active smart hydrogel layer 112 that is sandwiched between and attached to two polymer films 104a, 104b. Additional examples of such structures are shown in FIGS. 7A and 7B. The two polymer films 104 create an ultrasound resonator cavity 120 that exhibits similar behavior with regard to ultrasound (resonance absorption) as Applicant has described previously for other smart hydrogel resonator structures. When ultrasound with a frequency at or close to one of the resonance frequencies of the resonator cavity 120 is directed at the cavity, it will be subject to increased absorption of mechanical energy into the cavity to form a standing acoustic wave (resonance) inside the cavity 120. This energy is lost from the reflected/transmitted ultrasound wave. As the fraction or amount of ultrasound energy absorbed depends on the frequency separation between the ultrasound wave's frequency and the cavity's resonance frequency the amount of energy missing from the reflected/transmitted wave can be used to detect this frequency separation after appropriate calibration. As the resonance frequency of the cavity depends on the inter-film distance between the polymer films, any distance change, such as induced by the smart hydrogel's volume change in response to one or more analytes, will change the resonance frequency of the cavity. This in turn changes either the frequency spectrum or, if measured at a fixed ultrasound frequency, the absorption properties. Thus, by tracking the absorption loss of the ultrasound when transmitted/reflected through the cavity this can be used to remotely measure the analyte concentration. As all materials can be selected to be biocompatible, this is a very useful concept for building fully implantable sensors that can be remotely queried per ultrasound. As noted herein however, the concept is not limited to human or other animal implantable sensors, but can be applied in a wide variety of environments where remote sensing of analyte concentrations, pH, ionic strength or other parameters that can induce a volumetric transition in a smart hydrogel is desired.

The smart hydrogel structure within the microresonator sandwich structure is configured to be highly porous and is used to alter the inter-film distance, and thus the cavity's resonance frequency, in response to the presence of an analyte. As the smart hydrogel 112 is not directly involved in the resonance absorption process it can be as porous or with an as small scale as practical without impeding the functional principle of the resonance ultrasound absorption, resulting in an extremely fast reacting sensor. As the top and bottom films 104 (i.e., 104a, 104b) can also interconnect a plurality of high aspect ratio smart hydrogel structures, this can help to stabilize such hydrogel structures, and allow higher aspect ratio structures that would otherwise be practical.

Beneficial material and structural properties of the components are discussed, below, although the general principles described herein can also work with alternative materials and/or structures that may not necessarily provide each of the described beneficial properties.

Bottom Polymer Film (e.g., away from the ultrasound source): In case of reflective ultrasound measurements, in an embodiment, ideally the bottom polymer film layer 104b may be configured to be as reflective for ultrasound as possible (e.g., exhibiting a large acoustic impedance mismatch as compared to the surrounding solution). In case of transmissive ultrasound measurements, the bottom layer 104b may be configured with characteristics comparable to the top polymer film layer 104a. In either case the film layer 104 should adhere well to the smart hydrogel structures 112. If such is not naturally the case, a surface modification of the polymer film may be provided, e.g., as shown in FIG. 5A. If the reflection of the bottom layer 104b as provided by the selected polymer material itself is insufficient, an ultrasound reflective layer may be provided, e.g., as shown in FIGS. 5D-5F. Such a reflective layer can serve to introduce the desired strong acoustic impedance mismatch.

Top Polymer Film (towards the ultrasound source): The reflective properties of the top polymer film 104a may be a bit more involved as the top film at the same time needs to admit the ultrasound wave to the resonator cavity but also keep the ultrasound wave energy inside the cavity 120. For this the top polymer film 104a can be configured and selected to be partially but not completely transparent to the relevant ultrasound frequencies. If this partial transparency or sonolucence is not achieved by material selection alone, the top layer, as shown at 104a′ in FIG. 7A can be perforated by microstructuring techniques (as described in conjunction with FIG. 5B) to optimize the ultrasound transparency to a desired degree. For example, holes formed through the thickness of the polymer film layer 104a′ may serve to adjust ultrasound reflectance of the polymer film layer including such holes. Furthermore, one of the layers 104 should be transparent to the optical wavelength that is used for polymerization of the smart hydrogel structure(s) 112. A metal or other reflective coating or layer (e.g., 114 as in FIGS. 5D-5F) could be present, so long as an optically transparent path is available for polymerization of the smart hydrogel structures 112. Such can be achieved by structuring the metal layer 114 (e.g., see FIG. 5E). Such a structured metal layer may also optionally serve as a photomask during hydrogel polymerization.

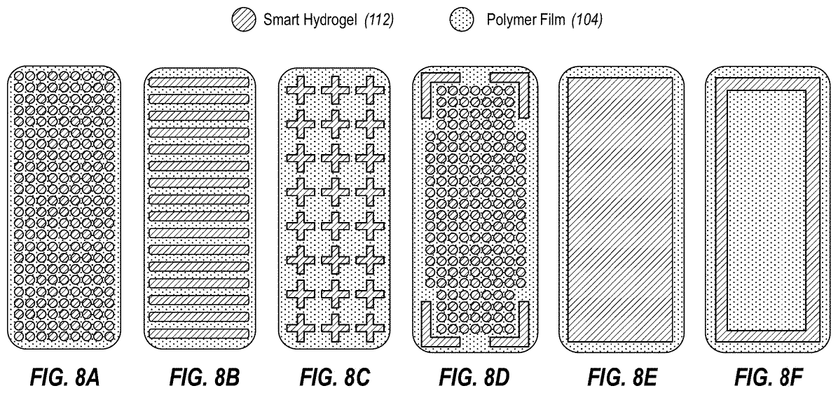

Structured Smart Hydrogel Layer: the smart hydrogel layer 112 is the active sensing component of the sensing structure. The smart hydrogel composition chosen is sensitive to the desired analyte and exhibits a volume change in the presence of the analyte. Furthermore, it is photopolymerizable, e.g., with a wavelength (e.g., UV) suitable for the suggested fabrication process. Ideally the smart hydrogel structures 112 have a very small critical dimension (i.e., the shortest distance to saturate the hydrogel by diffusion of analyte, such as the radius of a pillar, half the thickness of a wall, etc.). Small critical dimensions are balanced against providing sufficient thickness to stabilize the structure. Some non-limiting examples of such microresonator structures 100 are shown in FIGS. 8A-8F. The thickness of the hydrogel layer can be adjusted to the ultrasound frequency and its wavelength in the target analyte solution and should be approximately equal to multiple integers of half the wavelength. Optimal dimensions can be determined by simulations or experimental measurements. As a rough estimate a possible thickness or diameter may be about 300 μm in 1×PBS at a 4 MHz ultrasound query frequency. By way of example, FIG. 8A shows an array of pillars (e.g., similar to that of FIG. 7A). FIG. 8B shows a series of walls. FIG. 8C shows an array of geometric crosses. FIG. 8D shows a combination of a central array of pillars, with walls positioned near corners of the microresonator structure, similar to the configuration shown in FIG. 7B. FIG. 8E shows a porous continuous bulk layer (e.g., where the hydrogel is substantially transparent to the ultrasound query frequency). FIG. 8F shows a smart hydrogel structure configured as a peripheral outer wall, defining an internal free cavity. Such a structure may provide for improved resonator properties if the porous hydrogel is not sufficiently transparent to the ultrasound query frequency, e.g., due to interfacial reflectivity in the pores.

Closing remarks: while the structure described aims to create an ultrasound resonator cavity 120 including two polymer films 104, the described structure could alternatively be used to simply stabilize the smart hydrogel structure 112, rather than create a resonator cavity. In this case the top polymer film may be configured to be as transparent to ultrasound as possible and the smart hydrogel structures could act as ultrasound resonators in accordance with Applicant's previously described embodiments. Such a configuration would facilitate creation of larger aspect ratio resonators. Hybrid approaches where both a cavity and the smart hydrogel structure act as a resonator are also possible.

Fabrication: a non-limiting exemplary fabrication process with various optional and mitigation steps is outlined above in conjunction with FIGS. 4 and 5, which process can be used to fabricate the sandwich structures described herein. In an embodiment, the hydrogel structure could be fabricated in a mask-less process via two-photon polymerization 3D-lithography (see FIG. 5C). Alternative fabrication processes may also be possible, e.g., fabrication within a removeable microfluidic channel in a more manual process using techniques adapted from Applicant's previous publications.

### CONCLUSION

Any headings used herein are for organizational purposes only and are not meant to be used to limit the scope of the description or the claims.

Various aspects of the present disclosure, including devices, systems, and methods may be illustrated with reference to one or more embodiments or implementations, which are exemplary in nature. As used herein, the term “exemplary” means “serving as an example, instance, or illustration,” and should not necessarily be construed as preferred or advantageous over other embodiments disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” as well as variants thereof (e.g., “includes,” “has,” “involves,” “contains,” etc.), and similar terms as used herein, including within the claims, shall be inclusive and/or open-ended, shall have the same meaning as the word “comprising” and variants thereof (e.g., “comprise” and “comprises”), and do not exclude additional un-recited elements or method steps, illustratively. Unless defined otherwise, all technical and scientific terms used herein have the same meaning as commonly understood by one of ordinary skill in the art to which the present disclosure pertains.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition.

Disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure. Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Accordingly, the present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

What is claimed is:

1. A system for identifying one or more changes in a microresonator structure positioned within an in vivo or other environment, the system comprising:
a microresonator structure comprising a top polymer film layer, a bottom polymer film layer, and a smart hydrogel structure sandwiched therebetween, the top and bottom polymer film layers defining therebetween an ultrasound resonator cavity having at least one resonance frequency, wherein a height of the ultrasound resonator cavity changes due to volumetric expansion or contraction of the smart hydrogel structure sandwiched between the top and bottom polymer film layers in response to interaction of the smart hydrogel structure with one or more predefined analytes in the in vivo or other environment;
an ultrasound transducer for querying the microresonator structure within the in vivo or other environment at or near the resonance frequency of the ultrasound resonator cavity; and
a computer system in electrical communication with the ultrasound transducer, the computer system having one or more processors and being configured to:
receive, from the ultrasound transducer, ultrasound data as provided by query of the microresonator structure by the ultrasound transducer at or near the resonance frequency; and
determine, at the one or more processors, at least one of: (i) a change in amplitude or intensity of an ultrasound query wave or pulse as induced by interaction of the one or more predefined analytes with the smart hydrogel structure in the resonator cavity; (ii) a change in resonance frequency of the resonator cavity as induced by interaction of the one or more predefined analytes with the smart hydrogel structure; or (iii) a change in mean grayscale value (MGV) associated with the ultrasound data of the microresonator structure due to the change in the resonance frequency of the resonator cavity as induced by interaction of the one or more predefined analytes with the smart hydrogel structure.

2. The system as in claim 1, wherein the computer system is configured to determine the change in amplitude or intensity of an ultrasound query wave or pulse as induced by interaction of the one or more predefined analytes with the smart hydrogel structure in the resonator cavity.

3. The system as in claim 1, wherein the computer system receives, from the ultrasound transducer, the ultrasound data of the microresonator structure at a first time and at a second time, and wherein the computer system determines, at the one or more processors, a change in MGV, change in resonance frequency, or change in amplitude or intensity of the ultrasound wave or pulse associated with the smart hydrogel structure based on differences in the ultrasound data of the microresonator structure at the first time and at the second time.

4. The system as in claim 1, wherein the microresonator structure does not include any markers, contrast agents, or external connections.

5. The system as in claim 1, wherein the microresonator structure consists essentially of the smart hydrogel structure and the polymer top and bottom film layers.

6. The system as in claim 1, wherein the smart hydrogel structure in the resonator cavity is in the form of at least one of a bulk continuous sheet, one or more pillars, or one or more walls extending between the top and bottom polymer film layers.

7. The system as in claim 1, wherein the smart hydrogel structure within the microresonator structure has a thickness from 50 μm to 1000 μm.

8. The system as in claim 1, wherein the microresonator structure has a length and/or width that is from 0.1 mm to 20 mm.

9. The system as in claim 1, wherein the microresonator structure is biodegradable in vivo.

10. The system as in claim 1, wherein the system further comprises a control hydrogel positioned within the in vivo or other environment, the control hydrogel configured to not change in response to interaction with the one or more predefined analytes.

11. The system as in claim 1, wherein any change in dimension or volume of the smart hydrogel structure as a result of interaction with the one or more predefined analytes in the in vivo or other environment is not readily discernable in a generated ultrasound image.

12. A method for identifying one or more changes in a microresonator structure positioned within an in vivo or other environment, the method comprising:
providing a microresonator structure comprising a top polymer film layer, a bottom polymer film layer, and a smart hydrogel structure sandwiched therebetween, the top and bottom polymer film layers defining therebetween an ultrasound resonator cavity having a resonance frequency, wherein a height of the ultrasound resonator cavity changes due to volumetric expansion or contraction of the smart hydrogel structure sandwiched between the top and bottom polymer film layers in response to interaction of the smart hydrogel structure with one or more predefined analytes in the in vivo or other environment;
providing an ultrasound transducer for querying the microresonator structure within the in vivo or other environment at or near the resonance frequency of the ultrasound resonator cavity;
providing a computer system in electrical communication with the ultrasound transducer, the computer system having one or more processors and being configured to:
receive, from the ultrasound transducer, ultrasound data as provided by query of the microresonator structure by the ultrasound transducer at or near the resonance frequency; and
determine, at the one or more processors, at least one of: (i) a change in amplitude or intensity of an ultrasound query wave or pulse as induced by interaction of the one or more predefined analytes with the smart hydrogel structure in the resonator cavity: (ii) a change in resonance frequency of the resonator cavity as induced by interaction of the one or more predefined analytes with the smart hydrogel structure; (iii) a change in mean grayscale value (MGV) associated with the ultrasound data of the microresonator structure due to the change in the resonance frequency of the resonator cavity as induced by interaction of the one or more predefined analytes with the smart hydrogel structurer;
querying the microresonator structure with the ultrasound transducer in the in vivo or other environment, at or near a resonance frequency of the resonator cavity;
determining at least one of: (i) the change in amplitude or intensity of an ultrasound query wave or pulse as induced by interaction of the one or more predefined analytes with the smart hydrogel structure in the resonator cavity; (ii) the change in resonance frequency as induced by interaction of the one or more predefined analytes with the smart hydrogel structure in the in vivo or other environment; or (iii) the change in mean grayscale value (MGV) associated with the ultrasound data of the microresonator structure due to the change in the resonance frequency of the resonator cavity as induced by interaction of the one or more predefined analytes with the smart hydrogel structure in the in vivo or other environment; and
determining the presence of and/or a concentration of one or more predefined analytes based on the determination of (i), (ii) or (iii).

13. The method as in claim 12, wherein the microresonator structure is biodegradable in vivo, the method further comprising allowing the microresonator structure to biodegrade in vivo without retrieval thereof.

14. A microresonator structure comprising:
a top polymer film layer;
a bottom polymer film layer; and
a smart hydrogel structure sandwiched therebetween, the top and bottom polymer film layers defining therebetween an ultrasound resonator cavity having a resonance frequency, wherein the smart hydrogel is configured to provide a change in height of the ultrasound resonator cavity due to volumetric expansion or contraction of the smart hydrogel structure sandwiched between the top and bottom polymer film layers in response to interaction of the smart hydrogel structure with one or more predefined analytes in the in vivo or other environment.

15. The structure as in claim 14, wherein the microresonator structure does not include any markers, contrast agents, or external connections, or wherein the microresonator structure consists essentially of the smart hydrogel structure and the polymer top and bottom film layers.

16. The structure as in claim 14, wherein the smart hydrogel structure in the resonator cavity is in the form of at least one of a bulk continuous sheet, one or more pillars, or one or more walls extending between the top and bottom polymer film layers.

17. The structure as in claim 14, wherein the smart hydrogel structure within the microresonator structure has a thickness from 50 μm to 1000 μm and/or wherein the microresonator structure has a length and/or width that is from 0.1 mm to 20 mm.

18. The structure as in claim 14, wherein the microresonator structure is biodegradable in vivo.

19. The structure as in claim 14, wherein at least one of the polymer film layers includes an ultrasound reflective coating or layer.

20. The structure as in claim 14, wherein at least one of the polymer film layers includes holes formed therethrough to adjust ultrasound reflectance of the polymer film layer including such holes.
